(19) United States
(12) Patent Application Publication
(10) Pub. No.: US 2024/0417445 A1
(43) Pub. Date: Dec. 19, 2024

(54) METHOD FOR PRODUCING A LYOPHILIZED COMPOSITION COMPRISING POLYCLONAL IGM AND COMPOSITION OBTAINED

(71) Applicant: GRIFOLS WORLDWIDE OPERATIONS LIMITED, Dublin (IE)

(72) Inventors: Deepa SINGH, Research Triangle Park, NC (US); Anthony KLOS, Research Triangle Park, NC (US)

(21) Appl. No.: 18/672,438

(22) Filed: May 23, 2024

Related U.S. Application Data
(60) Provisional application No. 63/507,852, filed on Jun. 13, 2023.

(51) Int. Cl. C07K 16/06; A61K 9/19; A61K 47/18; A61K 47/22; A61K 47/26

(52) U.S. Cl. CPC C07K 16/065; A61K 9/19; A61K 47/183; A61K 47/22; A61K 47/26

(57) ABSTRACT

A lyophilized composition including polyclonal immunoglobulin M (IgM), including the steps of: a) providing an initial aqueous solution including IgM at a concentration between about 10 mg/mL and about 50 mg/mL, the polyclonal IgM being at least about 90% by weight of the total protein content of the composition; b) adding amino acids selected from the group consisting of proline, glycine, alanine, valine and hydroxyproline or a mixture thereof at a final concentration of about 0.15 M to about 0.45 M; polysorbate 80 (PS80) at a concentration between about 50 and about 200 ppm; and succinic acid at a concentration between about 1 mM and about 20 mM; and c) lyophilizing the IgM composition obtained in step b); wherein the lyophilized composition has a content of pentameric IgM higher than about 90% of the total IgM content, a content of IgM aggregates of less than about 1.5%.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### FIELD

The present invention refers to methods for obtaining a lyophilized composition comprising polyclonal immunoglobulin M (IgM), which is stable and can be used for many therapeutic indications.

### BACKGROUND

IgM is the second most abundant immunoglobulin, also called antibodies, in human plasma, after immunoglobulin G (IgG). IgM is the largest antibody, and it is the first antibody to appear in the response to initial exposure to an antigen, i.e. IgM is the predominant immunoglobulin isotype in the primary immune response.

A plasma-derived polyclonal IgM pharmaceutical composition suitable for human administration can be used to treat systemic antibiotic resistant bacterial infections (bacteremia), an area of unmet clinical need, though other indications may be considered. IgM circulates in plasma primarily in its pentameric form, comprised of 5 identical IgM monomer subunits connected by disulfide bonds.

The characteristics desired in IgM pharmaceutical compositions include high purity and high concentration plasma-derived polyclonal IgM. IgM products are not prevalent in part due to the difficulty associated with production of pure IgM solutions at concentrations suitable for therapeutic use. The product is a very large (900 kD) protein that can easily denature. As stated above, IgM in its pure form is a pentamer, but tends to self-associate into higher molecular weight species which may potentially pose immunogenic or other risks to patients. The high molecular weight species that form in the current liquid formulation can be categorized as oligomers and aggregates.

In addition, it is hard to maintain IgM stability after freezing and sublimation during lyophilization. Several problems occur during this process such as IgM self-association, increased opalescence of the product, long reconstitution time of the lyophilized cake and cracking the glass vials during the lyophilization process.

### SUMMARY

In view of the above, there is still the need to provide a process for obtaining a lyophilized composition comprising polyclonal IgM that overcomes said drawbacks. The present inventors have developed a process for obtaining lyophilized IgM compositions that surprisingly overcome the challenges typically associated with this protein. These problems are: (1) presence of high MW IgM species, (2) opalescence of the product, (3) long reconstitution times and (4) cracking the glass vials during the lyophilization process.

### DETAILED DESCRIPTION

The words “comprises/comprising” and the words “having/including” when used herein with reference to the present invention are used to specify the presence of stated features, integers, steps or components but do not preclude the presence or addition of one or more other features, integers, steps, components or groups thereof.

It should be appreciated by those skilled in the art that the specific embodiments disclosed herein should not be read in isolation, and that the present specification intends for the disclosed embodiments to be read in combination with one another as opposed to individually. As such, each embodiment may serve as a basis for modifying or limiting other embodiments disclosed herein.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “10 to 100” should be interpreted to include not only the explicitly recited values of 10 to 100, but also include individual value and sub-ranges within the indicated range. Thus, included in this numerical range are individual values such as 10, 11, 12, 13 . . . 97, 98, 99, 100 and sub-ranges such as from 10 to 40, from 25 to 40 and 50 to 60, etc. This same principle applies to ranges reciting only one numerical value, such as “at least 10”. Furthermore, such an interpretation should apply regardless of the breadth of the range or the characteristics being described.

As used herein, “about” means a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

As used in this specification and claims, the singular forms “a,” “an” and “the” include plural references unless the content clearly dictates otherwise.

In a first aspect, the present invention refers to a method for producing a lyophilized composition comprising polyclonal immunoglobulin M (IgM), comprising the steps of:
a) providing an initial aqueous solution comprising IgM at a concentration between about 10 mg/mL and about 50 mg/mL, the polyclonal IgM being at least about 90% by weight of the total protein content of the composition;
b) adding amino acids selected from the group consisting of proline, glycine, alanine, valine and hydroxyproline or a mixture thereof at a final concentration between about 0.15 M and about 0.45 M; polysorbate 80 (PS80) at a concentration between about 50 and about 200 ppm; and succinic acid at a concentration between about 1 mM and about 20 mM; and
c) lyophilizing the IgM composition obtained in step b);
   wherein said lyophilized composition has a content of pentameric IgM higher than about 90% of the total IgM content, a content of IgM aggregates of less than about 1.5%, and a content of IgM oligomers of less than about 7%.

As used herein, the term “lyophilized composition” means that the composition comprising polyclonal IgM is initially frozen and then water is removed by lowering the pressure. This terminology does not exclude additional drying steps that are present during the manufacturing process before the composition is filled with the final container. “Lyophilization” (freeze-drying) is a process for removing water, characterized by freezing the composition and then lowering the pressure and, optionally, heating to directly sublimate the frozen water in the composition from a solid phase to gas.

Preferably, said polyclonal IgM is human plasma-derived IgM.

As used herein, the term “plasma-derived” means that the IgM is derived from a plasma source which includes but is not limited to fresh-frozen plasma, non-fresh frozen plasma or a fraction thereof, such as an intermediate fraction produced using the fractionation schemes of Cohn et al., 1946 (E. J. Cohn et al., Preparation and Properties of Serum and Plasma Proteins. IV. A System for the Separation into Fractions of the Protein and Lipoprotein Components of Biological Tissues and Fluids. J. Am. Chem. Soc. 1946, 68, 3, 459-475) or Oncley et al., 1949 (J. L. Oncley et al., The separation of the antibodies, isoagglutinins, prothrombin, plasminogen and beta1-lipoprotein into subfractions of human plasma. J Am Chem Soc. 1949 February; 71(2):541-50) or a modification thereof or other plasma fraction. Accordingly, the term “plasma-derived” is not to be taken as being limited to plasma fractions derived using ethanolic precipitation methods.

Preferably, the pH of the initial aqueous solution comprising IgM is between about 3.8 and about 4.5, more preferably between about 4.0 and about 4.2.

Preferably, the initial solution comprising IgM of step a) has an IgM concentration between about 15 mg/mL and about 40 mg/mL, more preferably between about 17 mg/mL and about 36 mg/mL, even more preferably between about 18 mg/mL and about 33 mg/mL, even yet more preferably between 19 mg/mL and about 30 mg/mL.

Preferably, also in step a) polyclonal IgM is at least about 91%, 92%, 93%, 94%, 95%, 96%, 97%, 98%, 99%, or 100% by weight of the total protein content of the composition.

Preferably, in step b) the amino acid is proline, a mixture of glycine and alanine, a mixture of proline and glycine or a mixture of proline and alanine, more preferably an equimolar mixture of glycine and alanine, or an equimolar mixture of proline and glycine or an equimolar mixture of proline and alanine.

Preferably, in step b) the final concentration of the amino acid is between about 0.17 M and about 0.43 M, more preferably between about 0.19 M and about 0.41 M, even more preferably between about 0.21 M and about 0.39 M, even yet more preferably between about 0.22 M and about 0.35 M.

Preferably, in step b) the final concentration of polysorbate 80 (PS80) is between about 55 ppm and about 190 ppm, more preferably between about 60 ppm and about 180 ppm, even more preferably between about 70 ppm and about 150 ppm, even yet more preferably between about 80 ppm and about 120 ppm.

Preferably, in step b) the final concentration of succinic acid is between about 2 mM and 30 about 15 mM, more preferably between about 3 mM and about 10 mM, even more preferably between about 4 mM and about 8 mM.

Preferably, in step b) trehalose is further added at a concentration between about 5 mM and about 35 mM, more preferably between about 10 mM and about 30 mM, even more preferably between about 15 mM and about 25 mM, even yet more preferably between about 18 mM and about 22 mM.

Preferably, said lyophilized composition has a content of pentameric IgM higher than about 91%, 92%, 93%, 94%, 95%, 96%, 97%, 98%, 99% of the total IgM content.

Preferably, said lyophilized composition has a content of IgM aggregates of less than about 1.4%, 1.3%, 1.2%, 1.1%, 1.0%, 0.9%, 0.8%, 0.7%, 0.6%, 0.5%, 0.4%, 0.3%, or 0.2%.

Also preferably, the content of IgM oligomers in the lyophilized composition is of less than about 6%, 5%, 4%, 3%, 2%, or 1%.

Preferably, said lyophilized composition comprising lyophilized IgM is stable at least during 6, 7, 8, 9, 10, 11, 12, 13, 14, 15, 16, 17 or 18 months.

Preferably, the reconstitution time of said lyophilized composition is less than 9 minutes, 8 minutes, 7 minutes, 6 minutes, 5 minutes, 4 minutes, 3 minutes, or 2 minutes.

Preferably, the lyophilization method of step c) comprises the following steps:
heating the composition until about 38° C. to about 40° C., and holding at this temperature for about 2.5 h to about 3.5 h;
decreasing temperature until about −8° C. to about −3° C. during about 1 h to about 3 h, and holding at this temperature for about 4 h to about 6 h;
further decreasing temperature until about −43° C. and −48° C. (freezing ramp), and holding at this temperature for about 3 h to about 5 h;
increasing temperature until sublimation temperature at a rate of 10° C. to 20° C./h, and holding at this temperature as long as needed; and
holding at 30° C. desorption shelf temperature and 100 mTorr chamber pressure for up to 24 h until <0.5% water is left in the vial.

Preferably, the freezing ramp is carried out during about 1 h to about 6 h, and more preferably during about 1 h.

Prior to the lyophilization method the solution is filled into a container that is suitable for lyophilization, e.g., a glass vial.

After lyophilization, the pharmaceutical composition becomes a cake. Such a cake should be pharmaceutically acceptable. As used herein, a “lyophilized composition” refers to a non-collapsed solid drug product remaining after lyophilization that has certain desirable characteristics, e.g., pharmaceutically acceptable, long-term stability, a short reconstitution time, an elegant appearance and maintenance of the characteristics of the original solution upon reconstitution. The pharmaceutically acceptable cake can be solid, powder or granular material. The pharmaceutically acceptable cake may also contain up to five percent water by weight of the cake.

It should be appreciated by those skilled in the art that the specific embodiments disclosed within paragraphs [0013]-[0029] should not be read in isolation, and that the present specification intends for these embodiments to be disclosed in combination with other embodiments as opposed to being disclosed individually.

In another aspect, the present invention refers to a lyophilized composition comprising polyclonal IgM, wherein prior to lyophilization the initial aqueous solution comprises IgM at a concentration between about 15 mg/mL and about 50 mg/mL, the polyclonal IgM being at least about 90% by weight of the total protein content of the composition; further comprising amino acids selected from the group consisting of proline, glycine, alanine, valine and hydroxyproline or a mixture thereof at a final concentration of about 0.15 M to about 0.45 M; polysorbate 80 (PS80) at a concentration between about 50 and about 200 ppm; and succinic acid at a concentration between about 1 mM and about 20 mM; wherein said lyophilized composition has a content of pentameric IgM higher than about 90% of the total IgM content, a content of IgM aggregates of less than about 1.5%, and a content of IgM oligomers of less than about 7%.

Preferably, said polyclonal IgM is human plasma-derived IgM.

Preferably, the pH of the initial aqueous solution comprising IgM is between about 3.8 and about 4.5.

Preferably, prior to lyophilization said composition has an IgM concentration between about 15 mg/mL and about 40 mg/mL, more preferably between about 17 mg/mL and about 36 mg/mL, even more preferably between about 18 mg/mL and about 33 mg/mL, even yet more preferably between 19 mg/mL and about 30 mg/mL.

Preferably, prior to lyophilization in said composition polyclonal IgM is at least 91%, 92%, 93%, 94%, 95%, 96%, 97%, 98%, 99%, or 100% by weight of the total protein content of the composition.

Preferably, prior to lyophilization in said composition the amino acid is proline, a mixture of glycine and alanine, a mixture of proline and glycine or a mixture of proline and alanine, more preferably an equimolar mixture of glycine and alanine, or an equimolar mixture of proline and glycine or an equimolar mixture of proline and alanine.

Preferably, prior to lyophilization in said composition the final concentration of the amino acid is between about 0.17 M and about 0.43 M, more preferably between about 0.19 M and about 0.41 M, even more preferably between about 0.21 M and about 0.39 M, even yet more preferably between about 0.22 M and about 0.35 M.

Preferably, prior to lyophilization in said composition the final concentration of polysorbate 80 (PS80) is between about 55 ppm and about 190 ppm, more preferably between about 60 ppm and about 180 ppm, even more preferably between about 70 ppm and about 150 ppm, even yet more preferably between about 80 ppm and about 120 ppm.

Preferably, prior to lyophilization in said composition the final concentration of succinic acid is between about 2 mM and about 15 mM, more preferably between about 3 mM and about 10 mM, even more preferably between about 4 mM and about 8 mM.

Preferably, prior to lyophilization the composition further comprises trehalose at a concentration between about 5 mM and about 35 mM, more preferably between about 10 mM and about 30 mM, even more preferably between about 15 mM and about 25 mM, even yet more preferably between about 18 mM and about 22 mM.

Preferably, said lyophilized composition has a content of pentameric IgM higher than about 91%, 92%, 93%, 94%, 95%, 96%, 97%, 98%, 99% of the total IgM content.

Preferably, said lyophilized composition has a content of IgM aggregates of less than about 1.4%, 1.3%, 1.2%, 1.1%, 1.0%, 0.9%, 0.8%, 0.7%, 0.6%, 0.5%, 0.4%, 0.3%, or 0.2%.

Also preferably, the content of IgM oligomers in the lyophilized composition is of less than about 6%, 5%, 4%, 3%, 2%, or 1%.

Preferably, said lyophilized composition comprising lyophilized IgM is stable at least during 6, 7, 8, 9, 10, 11, 12, 13, 14, 15, 16, 17 or 18 months.

Preferably, the reconstitution time of said lyophilized composition is less than 9 minutes, 8 minutes, 7 minutes, 6 minutes, 5 minutes, 4 minutes, 3 minutes, or 2 minutes.

Although this disclosure is in the context of certain embodiments and examples, those skilled in the art will understand that the present disclosure extends beyond the specifically disclosed embodiments to other alternative embodiments and/or uses of the embodiments and obvious modifications and equivalents thereof. In addition, while several variations of the embodiments have been shown and described in detail, other modifications, which are within the scope of this disclosure, will be readily apparent to those of skill in the art based upon this disclosure.

It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes or embodiments of the disclosure. Thus, it is intended that the scope of the present disclosure herein disclosed should not be limited by the particular disclosed embodiments described above.

It should be understood, however, that this description, while indicating preferred embodiments of the disclosure, is given by way of illustration only, since various changes and modifications within the spirit and scope of the disclosure will become apparent to those skilled in the art.

It should be appreciated by those skilled in the art that the specific embodiments disclosed within paragraphs [0035]-[0049] should not be read in isolation, and that the present specification intends for these embodiments to be disclosed in combination with other embodiments as opposed to being disclosed individually. For example, each of the embodiments disclosed in paragraphs [0013]-[0029] is to be read as being explicitly combined with each of the embodiments in paragraphs [0035]-[0049], or any permutation of 2 or more of the embodiments disclosed therein.

The terminology used in the description presented herein is not intended to be interpreted in any limited or restrictive manner. Rather, the terminology is simply being utilized in conjunction with a detailed description of embodiments of the systems, methods and related components. Furthermore, embodiments may comprise several novel features, no single one of which is solely responsible for its desirable attributes or is believed to be essential to practicing the embodiments herein described.

### EXAMPLES

It should be readily apparent to one of ordinary skill in the art that the examples disclosed herein below represent generalized examples only, and that other arrangements and methods capable of reproducing the invention are possible and are embraced by the present invention.

### Example 1—Amino Acids as Excipients for Lyophilizing IgM

For the pre-lyophilized formulation, tested excipients for stabilizing lyophilized IgM include amino acids. The soluble amino acids include proline, hydroxyproline, glycine, alanine and valine.

The crystallinity of a formulation can be seen on a calorimeter. The formulations that show crystallization lose IgM pentamer content. Glycine and alanine form crystalline components and a loss of pentamers upon freezing and so were individually eliminated from formulations. However, surprisingly glycine and alanine in a 50-50 mix do not show crystallization. Of all the amino acids, proline yielded the highest concentration of pentamers and lowest concentration of aggregates.

When proline is used as the stabilizing excipient, the lengthy reconstitution time is the only one of the four problems listed above that occurs, and may take up to an hour for reconstituting 40 mL at 25 mg/mL IgM. Long reconstitution time is not preferred, but also is not an unusual problem in a clinical setting. This is typically mitigated by hospital pharmacist preparation in time to allow dosing as needed.

To reduce the reconstitution time, 20 mM trehalose was incorporated into the proline (230 mM) formulation. This reconstitution will take between 10 and 20 minutes after the lyophilization operation. In addition, this excipient combination resulted in reduced IgM aggregation compared to the formulation comprising proline (250 mM) alone. The 20 mM trehalose is a much lower concentration than is usually used to stabilize pharmaceutical formulations.

If the freezing step of IgM stabilized with proline and trehalose excipients is ramped for only an hour from −5° C. to −45° C., the reconstitution will take less than 5 minutes. If the freezing ramp takes longer, the reconstitutions will also be longer.

The proline/IgM or proline-trehalose/IgM formulations have good clarity and do not crack Type I vials.

The reconstitution time based on the freezing ramp is shown in Table 1.

TABLE 1
Reconstitution time of formulations using
different lyophilization freezing ramps.

| | Lyophilization | Reconstitution | |
|---|---|---|---|
| Excipients | freezing ramp | time | |
| Proline (250 mM) | Normal freezing | >1 | h |
| | ramp (3 h) | | |
| Proline (230 mM) + | Normal freezing | >10 | mins |
| trehalose (20 mM) | ramp (3 h) | | |
| Proline (230 mM) + | 1 h freezing ramp | <5 | min |
| trehalose (20 mM) | | | |

As shown in Table 1, addition of trehalose 20 mM reduces the reconstitution time from >1 h to >10 min and it is further reduced with a freezing ramp of 1 h.

### Example 2—an Equimolar Mix of Glycine and Alanine as Excipients for Lyophilizing IgM

When glycine and alanine are mixed in an equimolar ratio, there is no crystallinity detected by differential scanning calorimetry (DSC) when freezing a sample. These excipients allowed a high pentamer content in the IgM solution when combined.

Also, the combination shows low aggregation compared to the bulk, but not as low as proline. The reconstitution time is less than 5 minutes at any freezing ramp and the opalescence is higher in Nephelometric Turbidity Units (NTU) than the IgM formulations with proline or proline-trehalose. Finally, the glycine-alanine formulation can break glass vials during lyophilization even though there is no crystal formation in the bulk which may cause an increase in volume.

### Example 3—an Equimolar Mix of Glycine and Proline or an Equimolar Mix of Alanine with Proline as Excipients for Lyophilizing IgM

A proline excipient showed favorable results except for the long reconstitution time. Mixing proline with amino acids may resolve that problem without resorting to trehalose.

By mixing proline and alanine, or proline and glycine, no crystallization was observed, similar to mixtures of glycine and alanine. Proline with glycine and alanine also works.

By using proline-alanine or proline-glycine, the aggregation and opalescence problems are eliminated.

By using a long freezing ramp (−5° C. to −45° C. for 3 h to 4 h) the lyophilized IgM (with proline-glycine or proline-alanine) reconstitutes in less than 5 minutes with no cracked vials at a 40 mL fill of 25 mg/mL IgM in even the more crack-susceptible (thin-bottomed) vials (SGD Pharma, France).

### Example 4—Process for Obtaining Lyophilized IgM

IgM is concentrated to 20 mg/mL at pH 4.5-5.0. Once the desired concentration is achieved, 10 volumes of diafiltration are performed against either a succinate or a select formulation buffer at pH 4.0-4.2.

In the last step of ultrafiltration/diafiltration (UF/DF), IgM in the diafiltration buffer is concentrated to 40 mg/mL. After UF/DF, if diafiltered against the minimal succinate buffer, the material is recovered and formulated with chosen excipients at a final concentration of 200-250 mM. If diafiltered against a formulation buffer, the product is recovered and diluted to the target IgM concentration with additional formulation buffer. The formulated material is pH adjusted to 4.0-4.2, followed by a sterile filtration. The IgM concentration is 25 mg/mL.

Finally, the material is filled in vials, 40 mL fill at 25 mg/mL IgM, and placed in the lyophilizer.

Once the vials are lyophilized, they may be stored in appropriate stability chambers at 5° C. and at 30° C. Vials are taken out of the chambers at appropriate times and are reconstituted to characterize the product using SE-HPLC, clarity, and the reconstitution time assays of different IgM formulations.

The selected formulations used for the final containers are shown in Table 2. The pH of all formulations was in the range of 4.0 to 4.2.

TABLE 2
Selected formulations tested for IgM lyophilization.

| IgM (25 mg/mL) excipients | Succinic acid | Polysorbate 80 |
|---|---|---|
| Glycine (250 mM) | 5 mM | 100 ppm |
| Proline (250 mM) | 5 mM | 100 ppm |
| Proline (225 mM) & trehalose (25 mM) | 5 mM | 100 ppm |
| Proline (125 mM) & glycine (125 mM) | 5 mM | 100 ppm |
| Proline (125 mM) & alanine (125 mM) | 5 mM | 100 ppm |
| Glycine (125 mM) & alanine (125 mM) | 5 mM | 100 ppm |
| Glycine (225 mM) & trehalose (25 mM) | 5 mM | 100 ppm |
| Trehalose (250 mM) | 5 mM | 100 ppm |
| Hydroxyproline (250 mM) | 5 mM | 100 ppm |
| Valine (250 mM) | 5 mM | 100 ppm |

Formulations are lyophilized with a general lyophilization cycle, as shown in Table 3.

TABLE 3
Lyophilization cycle for obtaining lyophilized IgM using different formulations.

| | Time | S helf temp | | Pressure |
|---|---|---|---|---|
| Ramp 20° C. to 39° C. | | | | Ambient (>400 Torr) |
| Hold at 39° C. | NLT 3 h | 39° | C. | Ambient (>400 Torr) |
| Ramp to −5° C. | 1 h to 3 h | | | Ambient (>400 Torr) |
| Hold at −5° C. | NLT 5 h | −5° | C. | Ambient (>400 Torr) |
| Ramp to −45° C. | 1 h to 6 h | | | Ambient (>400 Torr) |
| Hold at −45° C. | NLT 4 h | −45° | C. | Ambient (>400 Torr) |
| Ramp to Sublimation temp | 10° C. to 20° C./ h | | | 100 mTorr |
| Hold at Sublimation | As long as | Sublime temp | | 100 mTorr |
| temp (−10° C. to +30° C.) | needed | | | |
| Ramp to desorption | 1 h to 6 h | NLT 30° | C. | 100 mTorr |
| | | | | (NLT 50 mTorr) |
| Hold at desorption temp | NLT 24 h | NLT 30° | C. | 100 mTorr |

### Example 5—Amino Acids Used to Stabilize IgM at 25 mg/mL

Five different formulations (glycine, glycine+alanine, proline, hydroxyproline, valine) of amino acids were tested to assess their suitability as lyophilized IgM formulations.

Starting with the bulk, Table 4 shows the SE-HPLC profile listing the population of IgM species in different bulk IgM formulations. The data in Tables 5 and 6 that follow, show what happens to the pentamers as the bulk is processed in the pre-freeze heating step (Table 5) and the bulk/pre-freeze heating/freezing steps (Table 6). The purpose of the experiment was to determine which of the amino acids allows cryostability of the IgM pentamer.

TABLE 4
IgM population profiles in different product bulk formulations.

| | IgM | IgM | IgM |
|---|---|---|---|
| | aggregates | oligomers | pentamer |
| IgM, (25 mg/mL) | (%) | (%) | (%) |
| Glycine | 2.2 | 3.8 | 94.0 |
| Glycine + Alanine | 2.1 | 3.7 | 94.1 |
| Proline | 1.2 | 3.1 | 95.7 |
| Hydroxyproline | 1.2 | 3.1 | 95.7 |
| Valine | 1.8 | 3.5 | 94.8 |

Samples of all five bulk formulations (using the same bulks as in Table 4) were heated in a water bath at 39° C. for 2 hours and then reanalyzed. Dissolution of the high molecular weight population was observed for all formulations, and Table 5 lists the % of each population pre- and post-heating. The effect of heating was mostly observed on the aggregates, rather than the oligomers. In all formulations, including glycine, heating led to a 2-3-fold reduction in the % high molecular weight aggregate and an increase in the % of the pentamer purity. The effect was more pronounced in the proline and hydroxyproline-based IgM formulations.

Table 5 shows the SE-HPLC profile of IgM for each of the 5 excipients, showing the effect of pre-freeze heating. The bulk values in Table 4 were compared to the IgM values when heated at 39° C. The heat step is displayed in bold.

TABLE 5
SE-HPLC profile of lyophilized IgM with each of
5 excipients showing the effect of post-reconstitution
heating. Data from heated samples in bold.

| | IgM aggregate | IgM oligomers | IgM pentamer |
|---|---|---|---|
| | % no heat | % no heat | % no heat |
| | step T4/ | step T4/ | step T4/ |
| IgM, 25 mg/mL | heat step | heat step | heat step |
| Glycine | 2.2/0.9 | 3.8/3.5 | 94.0/95.6 |
| Glycine + | 2.1/0.9 | 3.7/3.4 | 94.1/95.7 |
| Alanine | | | |
| Proline | 1.2/0.4 | 3.1/2.6 | 95.7/97.0 |
| Hydroxyproline | 1.2/0.4 | 3.1/2.5 | 95.7/97.0 |
| Valine | 1.8/0.7 | 3.5/2.9 | 94.8/96.3 |

### Effect of Freezing on IgM Populations in Different Formulations

After the pre-freeze heating step, the vials were chilled to −5° C. This allowed all of the vials in the lyophilizer to start at the same condition during the freezing ramp. Then the freezing temperature was ramped from −5° C. to −45° C. followed by freezing at −45° C. for all of the Ig formulations. After freezing, all of the vials were removed from the lyophilizer and thawed. The SE-HPLC percentage of each IgM population was analyzed.

Table 6 lists the SE-HPLC pentamer results for the bulk, pre-freeze heating, and post freeze-thaw of IgM.

TABLE 6
SE-HPLC profile of pentamer results for the bulk,
pre-freeze heating, and post freeze-thaw of IgM

| | IgM | IgM pentamer % | IgM pentamer % |
|---|---|---|---|
| | pentamer % | Pre-freeze | Post-freeze |
| IgM, 25 mg/mL | Bulk | heat step | step |
| Glycine | 94.0 | 95.6 | 92.4 |
| Glycine + Alanine | 94.1 | 95.7 | 95.7 |
| Proline | 95.7 | 97.0 | 97.1 |
| Hydroxyproline | 95.7 | 97.0 | 95.4 |
| Valine | 94.8 | 96.3 | 92.7 |

Only glycine mixed with alanine, and proline-based IgM formulations retain the purity of the pentamer population after the freezing operation.

Based on the freeze thaw studies, subsequent stability studies for different lyophilized IgM formulations included IgM in the glycine mixed with alanine formulation and the proline formulation. For comparison, a glycine-only based formulation was also included. Previous studies indicated that glycine is not a preferred excipient for lyophilized IgM (with post-lyophilization cracked vials, higher final container drug product turbidity, and higher oligomer and aggregate content in the final container drug product). Table 7 compares the stability of lyophilized IgM in different formulations over a course of 8 months. Table 7a, 7b and 7c lists the values for % pentamer, oligomers and aggregates as measured by SE-HPLC comparing the lyophilized glycine, glycine-alanine, and proline formulations. The initial data for all three formulations are also different, as proline appears to dissociate the aggregates in the bulk and also survives the freeze-drying process better than the other two formulations.

Based on the observation that heating reduces the prevalence of high MW IgM populations, a pre-freeze heating step was included in these and all subsequent lyophilization cycles. Once the vials were filled in a sterile environment, they were moved to the lyophilizer and were equilibrated at 20° C. for 0 to 30 minutes. Vials were then heated to a shelf temperature of 39° C. for about 2-4 h. The product temperature was approximately 37° C. during this step. A similar effect of heating was observed at 25 mg/mL and also higher IgM concentrations >25 mg/mL. Vials were then frozen and subjected to the remainder of the lyophilization cycle.

Table 7a shows IgM pentamer population in three lyophilized formulations up to 8 months at 5° C.

TABLE 7a

| IgM, 25 mg/mL | % pentamer 5° C. | | |
|---|---|---|---|
| Time points (months) | gly | gly/ala | proline |
| 0 | 91.4 | 93.6 | 95.5 |
| 0.25 | 91.2 | 93.4 | 95.5 |
| 0.5 | 91.3 | 93.3 | 95.4 |
| 1 | 91.0 | 93.4 | 95.4 |
| 3 | 90.5 | 93.0 | 95.3 |
| 4 | 90.0 | 93.2 | 95.5 |
| 6 | 89.5 | 92.9 | 95.6 |
| 8 | 88.8 | 92.7 | 95.1 |

Table 7b shows IgM oligomer population in three lyophilized formulations up to 8 months at 5° C.

TABLE 7b

| IgM, 25 mg/mL | % oligomers 5° C. | | |
|---|---|---|---|
| Time points (months) | gly | gly/ala | proline |
| 0 | 6.2 | 3.9 | 2.7 |
| 0.25 | 6.2 | 4.0 | 2.7 |
| 0.5 | 6.1 | 3.9 | 2.7 |
| 1 | 6.4 | 4.0 | 2.7 |
| 3 | 7.0 | 4.3 | 2.9 |
| 4 | 7.6 | 4.4 | 2.8 |
| 6 | 8.2 | 4.7 | 2.7 |
| 8 | 8.8 | 4.6 | 2.8 |

Table 7c shows IgM aggregate population in three lyophilized formulations up to 8 months at 5° C.

TABLE 7c

| IgM, 25 mg/mL | % aggregate 5° C. | | |
|---|---|---|---|
| Time points (months) | gly | gly/ala | proline |
| 0 | 1.1 | 1.1 | 0.4 |
| 0.25 | 1.1 | 1.1 | 0.4 |
| 0.5 | 1.1 | 1.1 | 0.4 |
| 1 | 1.1 | 1.2 | 0.4 |
| 3 | 1.1 | 1.3 | 0.4 |
| 4 | 1.1 | 1.1 | 0.4 |
| 6 | 1.1 | 1.1 | 0.4 |
| 8 | 1.1 | 1.1 | 0.4 |

In Table 7a, the % pentamer had a decrease of 3% for the glycine, 1% for glycine/alanine, and <0.5% for proline formulation over the course of 8 months. This stability for proline is reflected by no significant increase in the oligomer and the aggregate population. The 25 glycine and glycine/alanine formulations did show increases in the oligomers over 8 months.

### Stress Studies of Different IgM Formulations for 6 Months

For stress-related studies, these lyophilized formulations were also stored at 30° C. for 6 months and the data is presented in Tables 8a, 8b and 8c listing the % pentamer, oligomer an aggregate as measured by SE-HPLC for the 3 lyophilized-formulations.

Table 8a shows IgM pentamer population in three lyophilized formulations up to 6 months at 30° C.

TABLE 8a

| IgM, 25 mg/mL | % pentamer 30° C. | | |
|---|---|---|---|
| Time points (months) | gly | gly/ala | proline |
| 0 | 91.4 | 93.6 | 95.5 |
| 0.25 | 88.3 | 92.1 | 95.2 |
| 0.5 | 85.6 | 91.2 | 95.1 |
| 1 | 82.3 | 90.5 | 94.9 |
| 3 | 72.4 | 86.4 | 93.8 |
| 4 | 67.8 | 85.4 | 94.2 |
| 6 | 63.4 | 82.9 | 93.8 |

Table 8b shows IgM oligomer population in three lyophilized formulations up to 6 months at 30° C.

TABLE 8b

| IgM, 25 mg/mL | | | |
|---|---|---|---|
| Time points | % oligomers 30° C. | | |
| (months) | gly | gly/ala | proline |
| 0 | 6.2 | 3.9 | 2.7 |
| 0.25 | 9.0 | 5.3 | 2.9 |
| 0.5 | 11.6 | 6.0 | 2.9 |
| 1 | 14.7 | 6.8 | 3.1 |
| 3 | 23.1 | 11.0 | 4.4 |
| 4 | 26.5 | 12.2 | 3.9 |
| 6 | 30.9 | 14.5 | 4.2 |

Table 8c shows IgM aggregate population in three lyophilized formulations up to 6 months at 30° C.

TABLE 8c

| IgM, 25 mg/mL | % aggregate 30° C. | | |
|---|---|---|---|
| Time points (months) | gly | gly/ala | proline |
| 0 | 1.1 | 1.1 | 0.4 |
| 0.25 | 1.1 | 1.1 | 0.4 |
| 0.5 | 1.3 | 1.2 | 0.4 |
| 1 | 1.4 | 1.2 | 0.4 |
| 3 | 3.3 | 1.4 | 0.5 |
| 4 | 4.3 | 1.1 | 0.6 |
| 6 | 4.4 | 1.2 | 0.6 |

As shown in Tables 8a, 8b and 8c, proline-based IgM formulations have higher stability as compared to the other two formulations. The oligomer species in the proline formulation increases by less than 2-fold over 6 months compared to roughly 5-fold and 4-fold for the glycine and glycine mixed with alanine formulations respectively. The aggregate species increase by 4-fold in the glycine formulation, whereas no significant increase is seen for glycine mixed with alanine and the proline formulations.

### Clarity of Lyophilized IgM Formulations

The clarity of the three lyophilized formulations was assessed using A450 nm measurements. It was observed that the proline formulation had more clarity post-formulation, with data presented in Table 9 showed that the clarity was retained post-lyophilization and during storage. At 5° C., the clarity of the glycine and glycine mixed with alanine formulations were 3-fold lower than the proline formulation. Under stressed conditions at 30° C., the proline formulation again maintains a higher clarity than the other two lyophilized formulations.

Table 9 shows the clarity of the three IgM lyophilized formulations post-reconstitution over the course of 8 months as measured by A450 nm.

TABLE 9

| IgM, 25 mg/mL | | | | | | |
|---|---|---|---|---|---|---|
| Time points | Clarity 5° C. (A450 nm) | | | Clarity 30° C. (A450 nm) | | |
| (months) | gly | gly/ala | proline | gly | gly/ala | proline |
| 0.5 | 0.040 | 0.048 | 0.017 | 0.055 | 0.063 | 0.017 |
| 1 | 0.042 | 0.040 | 0.019 | 0.055 | 0.049 | 0.019 |
| 3 | 0.047 | 0.051 | 0.021 | 0.074 | 0.078 | 0.022 |
| 4 | 0.043 | 0.042 | 0.014 | 0.068 | 0.056 | 0.023 |
| 6 | 0.046 | 0.053 | 0.014 | 0.073 | 0.068 | 0.048 |

| | | | | |
|---|---|---|---|---|
| 8 | 0.050 | 0.047 | 0.016 | Not available |

### Reconstitution Time of Different IgM Formulations

Proline-based lyophilized formulations had longer reconstitution times after lyophilization. Addition of 25 mM trehalose to the proline-based formulations decreased the reconstitution time. Speeding up the freezing ramp made the reconstitution go faster for both proline and proline/trehalose lyophilized formulations.

Table 10 shows the effect of freezing rate on the reconstitution of proline-based IgM formulations.

TABLE 10

| | | |
|---|---|---|
| Formulations | Slow Freezing (3 h) | Faster freezing (1 h) |

| | | | | |
|---|---|---|---|---|
| Proline (250 m) | >2 | hours | >2 | hour |

| | |
|---|---|
| 15-20 particles | 1-2 particles |
| still floating | still floating |

| | | | | |
|---|---|---|---|---|
| Proline (225 mM) + | <14 | min | <3 | min |
| Trehalose (25 mM) | | | | |

### Stability of Trehalose Containing Lyophilized Formulations

Due to the effect of trehalose on the reconstitution time of proline lyophilized formulations, the stability of trehalose-containing lyophilized formulations was investigated. SE-HPLC data up to six months is presented in Table 11. As shown in Table 11a, the pentamer stability is maintained over 6 months, which is similar to non-trehalose based formulations. The data for the oligomer and aggregates population at 5° C. is presented in Table 11b and 11c.

Table 11a shows IgM pentamer population in two lyophilized formulations up to 6 months at 5° C.

TABLE 11a

% pentamer, 5° C.

| IgM, 25 mg/mL | Gly (115 mM) + ala | |
|---|---|---|
| Time points | (115 mM) + trehalose | Proline (225 mM) + |
| (months) | (25 mM) | trehalose (25 mM) |
| 1 | 93.6 | 95.2 |
| 2 | 93.5 | 95.2 |
| 3 | 93.2 | 94.9 |
| 4 | 94.1 | 96.6 |
| 6 | 94.2 | 95.7 |

Table 11b shows IgM oligomer population in two lyophilized formulations up to 6 months at 5° C.

Table

TABLE 11b

| | |
|---|---|
| IgM, 25 mg/mL | % oligomer, 5° C. |

| Time points | gly + ala + | proline + |
|---|---|---|
| (months) | trehalose | trehalose |
| 1 | 4.2 | 3.0 |
| 2 | 4.2 | 3.1 |
| 3 | 4.2 | 3.1 |
| 4 | 4.3 | 2.5 |
| 6 | 4.2 | 3.1 |

11c shows IgM aggregate population in two lyophilized formulations up to 6 months at 5° C.

TABLE 11c

| | |
|---|---|
| IgM, 25 mg/mL | % aggregate, 5° C. |

| Time points | gly + ala + | proline + |
|---|---|---|
| (months) | trehalose | trehalose |
| 1 | 0.98 | 0.42 |
| 2 | 1.01 | 0.43 |
| 3 | 1.02 | 0.44 |
| 4 | 0.96 | 0.11 |
| 6 | 0.98 | 0.39 |

Table 11d shows IgM pentamer population in two lyophilized formulations up to 6 months at 30° C.

TABLE 11d

| | |
|---|---|
| IgM, 25 mg/mL | % Mono-pentamer, 30° C. |

| Time points | gly + ala + | proline + |
|---|---|---|
| (months) | trehalose | trehalose |
| 1 | 92.1 | 95.0 |
| 2 | 91.5 | 94.8 |
| 3 | 90.9 | 94.2 |
| 6 | 90.6 | 94.8 |

Table 11e shows IgM oligomer population in two lyophilized formulations up to 6 months at 30° C.

TABLE 11e

| | |
|---|---|
| IgM, 25 mg/mL | % oligomer, 30° C. |

| Time points | gly + ala + | proline + |
|---|---|---|
| (months) | trehalose | trehalose |
| 1 | 5.43 | 3.39 |
| 2 | 5.97 | 3.54 |
| 3 | 6.35 | 3.63 |
| 6 | 7.65 | 3.92 |

Table 11f shows IgM aggregate population in two lyophilized formulations up to 6 months at 30° C.

TABLE 11f

| | |
|---|---|
| IgM, 25 mg/mL | % aggregate, 30° C. |

| Time points | gly + ala + | proline + |
|---|---|---|
| (months) | trehalose | trehalose |
| 1 | 1.13 | 0.43 |
| 2 | 1.28 | 0.44 |
| 3 | 1.23 | 0.47 |
| 6 | 1.14 | 0.46 |

Stability Trial Data of the Proline (230 mM)+Trehalose (20 mM) with 25 mg/mL IgM and Also the Glycine (125 mM) and Alanine (125 mM) Excipients with 25 mg/mL IgM (with No Trehalose)

These are the candidates selected for stability trials of 25 mg/mL IgM. Both formulations also contain succinic acid (nominally 5 mM) and PS80 (nominally 100 ppm). First the initial data will be shown in Table 12. These come from the same batch of IgM. Then the time data of each excipient will be shown for the proline-trehalose excipients and the glycine-alanine excipients over the course of 18 months at 5 and 30° C. and 6 months at 40° C. IgM binding to bacterial antigens (P. aeruginosa and K. pneumoniae) as well as IgM activity (binding to E. coli LPS+subsequent complement activation) during storage was also tested.

Table 12 shows initial post-lyophilization IgM formulations tracked for stability.

TABLE 12

| | Proline trehalose | glycine-alanine |
|---|---|---|
| | IgM Initial | IgM initial |
| Cake moisture (%) | 0.09 | 0.46 |
| Reconstitution time (min) | 9 | 8.5 |
| A 450 nm | 0.0103 | 0.0043 |
| Clarity | Opalescent | Opalescent |
| Visible particulates | none | none |
| Biuret (mg/mL) | 23.92 | 23.28 |
| SE-HPLC | | |
| IgM aggregate % | <0.100 | <0.100 |
| IgM oligomer % | 1.302 | 2.985 |
| lgM pentamer % | 98.165 | 96.461 |
| lgM < pentamer % | 0.500 | 0.464 |
| Antigen Binding - | | |
| Activity (U/mL) | | |
| anti-P. Aeruginosa - ELISA | 90.8 | 91.9 |
| anti-K pneumoniae LPS - | 103.8 | 106.5 |
| ELISA | | |
| anti-E coli LPS - Activity | 86.5 | 97.9 |
| Osmolality (mOsmol/kg) | 250 | 248 |
| pH | 4.03 | 4.05 |
| Succinic acid (mM) | 6.1 | Not tested |
| Glycine (mM) | Not tested | 111.7 |
| PS80 μg/mL | 91.21 | 87.35 |
| Turbidity (NTU) | 4.41 | 8.66 |
| Viscosity (cps) | 2.07 | 1.8 |

Table 13a shows 18 months post-lyophilization proline-trehalose IgM formulations put on 500 stability.

TABLE 13a

| Proline trehalose @ 5° C. | Initial | 3 month | 6 month | 9 month | 12 month | 18 month |
|---|---|---|---|---|---|---|
| Cake moisture (%) | 0.09 | 0.13 | 0.09 | 0.14 | 0.13 | 0.10 |
| Reconstitution time (min) | 9 | 8.5 | 5.5 | 2.9 | 3.1 | 3.5 |
| A 450 nm | 0.0103 | 0.0077 | 0.0091 | 0.0062 | 0.006 | 0.0073 |
| Clarity | Opalescent | Opalescent | Opalescent | Opalescent | Opalescent | Clear |
| Particulates (visible) | none | none | none | none | none | none |
| pH | 4.03 | 4.03 | 3.98 | 4.00 | 4.00 | 3.96 |
| Turbidity (NTU) | 4.41 | 4.53 | 4.85 | 4.50 | 4.44 | 4.70 |
| MFI | | | | | | |
| Particles/mL >2 μm | 1213.98 | Not tested | Not tested | Not tested | 1908.04 | Not tested |
| Light Obscuration | | | | | | |
| >10 μm Particles/mL | 1.2 | Not tested | Not tested | Not tested | 20.67 | Not tested |
| >25 μm Particles/mL | 0.07 | Not tested | Not tested | Not tested | 0.00 | Not tested |
| SE-HPLC | | | | | | |
| IgM aggregate % | <0.100 | <0.100 | <0.100 | <0.100 | <0.100 | <0.100 |
| IgM oligomer % | 1.302 | 1.432 | 1.351 | 1.484 | 1.462 | 1.590 |
| IgM pentamer % | 98.165 | 97.966 | 98.123 | 97.994 | 97.990 | 97.841 |
| IgM < pentamer % | 0.500 | 0.538 | 0.526 | 0.522 | 0.548 | 0.569 |
| Antigen Binding - Activity (U/mL) | | | | | | |
| anti-P. Aeruginosa - ELISA | 90.8 | 89.2 | 82.2 | 94.9 | 101.3 | 75.8 |
| anti-K. pneumoniae LPS - ELISA | 103.8 | 83.4 | 101.0 | 78.7 | 82.1 | 112.4 |
| anti-E. coli LPS - Activity | 86.5 | 75.0 | 71.2 | 78.4 | 89.7 | 87.6 |

The proline/trehalose IgM formulation is consistent when held at 5° C. for up to 18 months according to all test methods.

Table 13b shows 18 months post-lyophilization proline-trehalose IgM formulations put on 3000 stability.

TABLE 13b

| Proline trehalose @30° C. | Initial | 3 month | 6 month | 9 month | 12 month | 18 month |
|---|---|---|---|---|---|---|
| Cake moisture (%) | 0.09 | 0.21 | 0.13 | 0.16 | 0.17 | 0.15 |
| Reconstitution time (min) | 9 | 8.5 | 5.5 | 2.9 | 3.1 | 3.5 |
| A 450 nm | 0.0103 | 0.0086 | 0.0081 | 0.0068 | 0.01 | 0.0088 |
| Clarity | Opalescent | Opalescent | Opalescent | Opalescent | Opalescent | Clear |
| Particulates | none | none | none | none | none | none |
| pH | 4.03 | 4.02 | 3.98 | 4.01 | 3.97 | 3.97 |
| Turbidity (NTU) | 4.41 | 4.74 | 4.89 | 4.63 | 4.61 | 4.84 |
| MFI | | | | | | |
| Particles/mL >2 μm | 1,213.98 | Not tested | Not tested | Not tested | 1,786.60 | Not tested |
| Light Obscuration | | | | | | |
| >10 μm Particles/mL | 1.20 | Not tested | Not tested | Not tested | 1.67 | Not tested |
| >25 μm Particles/mL | 0.07 | Not tested | Not tested | Not tested | 0.00 | Not tested |
| SE-HPLC | | | | | | |
| IgM aggregate (%) | <0.100 | <0.100 | <0.100 | <0.100 | <0.100 | <0.100 |
| IgM oligomer (%) | 1.302 | 2.104 | 2.760 | 2.991 | 3.264 | 3.990 |
| IgM pentamer (%) | 98.165 | 97.347 | 96.669 | 96.510 | 96.197 | 95.431 |
| IgM < pentamer (%) | 0.500 | 0.549 | 0.538 | 0.499 | 0.539 | 0.558 |
| Antigen Binding - Activity U/mL | | | | | | |
| anti-P. Aeruginosa - ELISA | 90.8 | 92.6 | 78.8 | 81.9 | 88.4 | 80.2 |
| anti-K. pneumoniae LPS - ELISA | 103.8 | 79.5 | 106.5 | 87.6 | 81.4 | 94.8 |
| anti-E. coli LPS - Activity | 86.5 | 85.5 | 79.4 | 79.5 | 90.5 | 89.1 |

At 30° C., the proline/trehalose IgM formulation is largely consistent over time. A small increase in oligomer content is observed, but there does not appear to be an effect on clarity or IgM function.

Table 13c shows 6 months post-lyophilization proline-trehalose IgM formulations put on 40° C. stability.

TABLE 13c

| Proline trehalose @ 40° C. | Initial | 3 month | 6 month |
|---|---|---|---|
| Cake moisture (%) | 0.09 | 0.12 | 0.10 |
| Reconstitution time (min) | 9 | 8.5 | 7.5 |
| A 450 nm | 0.0103 | 0.0099 | 0.0093 |
| Clarity | Opalescent | Opalescent | Opalescent |
| Particulates | none | none | none |
| pH | 4.03 | 4.02 | 3.97 |
| Turbidity (NTU) | 4.41 | 4.73 | 5.19 |
| SE-HPLC | | | |
| IgM aggregate (%) | <0.100 | <0.100 | <0.100 |
| IgM oligomer (%) | 1.302 | 3.633 | 5.455 |
| IgM pentamer (%) | 98.165 | 95.826 | 93.921 |
| IgM < pentamer (%) | 0.500 | 0.541 | 0.563 |
| Antigen Binding - | | | |
| Activity (U/mL) | | | |
| anti-P. Aeruginosa - ELISA | 90.8 | 92.8 | 84.2 |
| anti-K. pneumoniae LPS - | 103.8 | 87.2 | 101.2 |
| ELISA | | | |
| anti-E. coli LPS - Activity | 86.5 | 86.3 | 86.5 |

At 40° C., a more pronounced increase in oligomer is detected as well as a slight increase in turbidity IgM function appears to be fully retained.

Table 14a shows 18 months post-lyophilization, post-reconstitution glycine-alanine IgM formulations put on 500 stability.

TABLE 14a

| Glycine Alanine 5° C. | Initial | 3 month | 6 month | 9 month | 12 month | 18 month |
|---|---|---|---|---|---|---|
| Cake moisture (%) | 0.46 | 0.51 | 0.51 | 0.65 | 0.50 | 0.51 |
| Reconstitution time (min) | 2.5 min | 4.0 | 3.0 | 2.2 | 3.2 | 1.9 |
| A 450 nm | 0.0043 | 0.0148 | 0.0145 | 0.013 | 0.012 | 0.0117 |
| Clarity | Opalescent | Opalescent | Opalescent | Opalescent | Opalescent | Opalescent |
| Particulates | none | none | none | none | none | none |
| pH | 4.05 | 4.02 | 3.95 | 3.99 | 3.98 | 3.98 |
| Turbidity (NTU) | 8.66 | 8.92 | 9.66 | 8.75 | 8.88 | 9.61 |
| MFI | | | | | | |
| Particles/mL >2 μm | 156.86 | not tested | not tested | not tested | 610.15 | not tested |
| Light Obscuration | | | | | | |
| >10 μm Particles | 1.27 | not tested | not tested | not tested | 8.67 | not tested |
| >25 μm Particles | 0.07 | not tested | not tested | not tested | 0.00 | not tested |
| SE-HPLC | | | | | | |
| IgM aggregate % | <0.1 | <0.1 | <0.1 | <0.1 | <0.1 | 0.137 |
| IgM oligomer % | 2.985 | 3.374 | 4.192 | 4.873 | 4.782 | 5.503 |
| IgM pentamer % | 96.461 | 96.039 | 95.179 | 94.583 | 94.661 | 93.810 |
| IgM < pentamer % | 0.464 | 0.520 | 0.563 | 0.489 | 0.499 | 0.549 |
| Antigen Binding - Activity (U/mL) | | | | | | |
| anti-P. Aeruginosa - ELISA | 91.9 | 96.1 | 79.8 | 72.5 | 81.3 | 81.7 |
| anti-K. pneumoniae LPS - ELISA | 106.5 | 82.7 | 89.1 | 75.5 | 79.2 | 90.0 |
| anti-E. coli LPS - Activity | 97.9 | 79.2 | 95.8 | 86.3 | 87.8 | 82.6 |

At 5° C., the glycine/alanine IgM formulation is characterized by a small increase in IgM oligomer and solution turbidity over 18 months. No trend in IgM activity was observed.

Table 14b shows 18 months post-lyophilization, post-reconstitution glycine-alanine IgM formulations put on 3000 stability.

TABLE 14b

| Glycine-Alanine 30° C. | Initial | 3 month | 6 month | 9 month | 12 month | 18 month |
|---|---|---|---|---|---|---|
| Cake moisture (%) | 0.46 | 0.62 | 0.71 | 0.64 | 0.59 | 0.56 |
| Reconstitution time (min) | 2.5 | 5 | 4.5 | 2.7 | 3.1 | 1.8 |
| A 450 nm | 0.0043 | 0.0147 | 0.0168 | 0.0158 | 0.0160 | 0.0176 |
| Clarity | Opalescent | Opalescent | Opalescent | Opalescent | Opalescent | Opalescent |
| Particulates | none | none | none | none | none | none |
| pH | 4.05 | 4.02 | 3.97 | 3.99 | 3.97 | 3.97 |
| Turbidity (NTU) | 8.66 | 9.81 | 11.3 | 10.8 | 12 | 14.7 |
| MFI | | | | | | |
| Particles/mL >2 μm | 156.86 | not tested | not tested | not tested | 4253.35 | not tested |
| Light Obscuration | | | | | | |
| >10 μm Particles | 1.27 | not tested | not tested | not tested | 3.33 | not tested |
| >25 μm Particles | 0.07 | not tested | not tested | not tested | 0 | not tested |
| SE-HPLC | | | | | | |
| IgM aggregate % | <0.1 | 0.2800 | 0.4060 | 0.8930 | 2.6120 | 7.046 |
| IgM oligomer % | 2.985 | 13.350 | 19.196 | 22.492 | 26.521 | 30.869 |
| IgM pentamer | 96.461 | 85.846 | 79.894 | 76.110 | 70.354 | 61.571 |
| IgM < pentamer | 0.464 | 0.525 | 0.505 | 0.506 | 0.513 | 0.515 |
| Antigen Binding - Activity U/mL | | | | | | |
| anti-P. Aeruginosa - ELISA | 91.9 | 94.8 | 78.3 | 89.2 | 92..0 | 78.8 |
| anti-K. pneumoniae LPS - ELISA | 106.5 | 87.2 | 99.2 | 86.2 | 79.6 | 83.7 |
| anti-E. coli LPS - Activity | 97.9 | 77.9 | 82.6 | 103.9 | 101 | 105.2 |

At 30° C., IgM aggregate and oligomer content as well as solution turbidity and particle counts increase during storage. There does not appear to be a clear trend in IgM activity, however.

Table 14c shows 6 months post-lyophilization glycine-alanine IgM formulations put on 40° C. stability.

TABLE 14c

| Glycine Alanine 40° C. | Initial IgM | 3 mo. 40° C. | 6 mo. 40° C. |
|---|---|---|---|
| Cake moisture (%) | 0.46 | 0.76 | 0.61 |
| Reconstitution time (min) | 2.5 | 4 | 3 |
| A 450 nm | 0.0043 | 0.0191 | 0.0312 |
| Clarity | Opalescent | Opalescent | Opalescent |
| Particulates | none | none | none |
| pH | 4.05 | 4.01 | 3.99 |
| Turbidity (NTU) | 8.66 | 12.6 | 18 |
| SE-HPLC | | | |
| IgM aggregate % | <0.1 | 3.816 | 13.000 |
| IgM oligomer % | 2.985 | 29.103 | 31.519 |
| IgM pentamer % | 96.461 | 66.537 | 54.963 |
| IgM < pentamer % | 0.464 | 0.543 | 0.518 |
| Antigen Binding - | | | |
| Activity (U/mL) | | | |
| anti-P. Aeruginosa - ELISA | 91.9 | 96.3 | 91.4 |
| anti-K. pneumoniae LPS - | 106.5 | 90.9 | 115.3 |
| ELISA | | | |
| anti-E. coli LPS - Activity | 97.9 | 91.2 | 103.4 |

At 40° C., IgM aggregate and oligomer content as well as turbidity increase during storage. There does not appear to be a clear trend in IgM activity, however.

What is claimed is:

1. A method for producing a lyophilized composition comprising polyclonal immunoglobulin M (IgM), comprising:
a) providing an initial aqueous solution comprising IgM at a concentration between about 10 mg/mL and about 50 mg/mL, the polyclonal IgM being at least about 90% by weight of the total protein content of the composition;
b) adding amino acids selected from the group consisting of proline, glycine, alanine, valine and hydroxyproline or a mixture thereof at a final concentration of about 0.15 M to about 0.45 M; polysorbate 80 (PS80) at a concentration between about 50 and about 200 ppm; and succinic acid at a concentration between about 1 mM and about 20 mM; and
c) lyophilizing the IgM composition obtained in said b);
wherein said lyophilized composition has a content of pentameric IgM higher than about 90% of the total IgM content, a content of IgM aggregates of less than about 1.5%, and a content of IgM oligomers of less than about 7%.

2. The method according to claim 1, wherein said polyclonal IgM is human plasma-derived IgM.

3. The method according to claim 1, wherein the pH of the initial aqueous solution comprising IgM is between about 3.8 and about 4.5.

4. The method according to claim 1, wherein IgM in said a) is at a concentration between about 15 mg/mL and about 40 mg/mL.

5. The method according to claim 1, wherein in said a) polyclonal IgM is at least about 95% by weight of the total weight of the composition.

6. The method according to claim 1, where in said b) the amino acid is proline, a mixture of glycine and alanine, a mixture of proline and glycine or a mixture of proline and alanine.

7. The method according to claim 6, said mixture of glycine and alanine is an equimolar mixture of glycine and alanine.

8. The method according to claim 6, said mixture of proline and glycine is an equimolar mixture of proline and glycine.

9. The method according to claim 6, said mixture of proline and alanine is an equimolar mixture of proline and alanine.

10. The method according to claim 1, wherein in said b) the final concentration of the amino acid is between about 0.17 M and about 0.43 M.

11. The method according to claim 1, wherein in said b) the final concentration of PS80 is between about 55 ppm and about 190 ppm.

12. The method according to claim 1, wherein in said b) the final concentration of succinic acid is between about 2 mM and about 15 mM.

13. The method according to claim 1, wherein in said b) trehalose is further added at a concentration between about 5 mM and about 35 mM.

14. The method according to claim 1, wherein said lyophilized composition has a content of pentameric IgM higher than about 95% of the total IgM content.

15. The method according to claim 1, wherein said lyophilized composition has a content of IgM aggregates of less than about 1.0%.

16. The method according to claim 1, wherein said lyophilized composition has a content of IgM oligomers of less than about 5%.

17. The method according to claim 1, wherein said lyophilized composition is stable at least during 6 months.

18. The method according to claim 1, wherein the reconstitution time is less than 10 minutes.

19. The method according to claim 1, wherein the lyophilization method comprises:
heating the composition until about 38° C. to about 40° C., and holding at this temperature for about 2.5 h to about 3.5 h;
decreasing temperature until about −8° C. to about −3° C., and holding at this temperature for about 4 h to about 6 h;
further decreasing temperature until about −43° C. and −48° C. (freezing ramp) during about 1 h to about 6 h, and holding at this temperature for about 3 h to about 5 h;
increasing temperature until sublimation temperature at a rate of 10° C. to 20° C./h, and holding at this temperature as long as needed;
holding at 30° C. desorption shelf temperature and 100 mTorr chamber pressure for up to 24 h until <0.5% water is left in the vial.

20. A composition comprising lyophilized IgM wherein prior to lyophilization the initial aqueous solution comprises IgM at a concentration between about 15 mg/mL and about 50 mg/mL, the polyclonal IgM being at least about 90% by weight of the total protein content of the composition; further comprising amino acids selected from the group consisting of proline, glycine, alanine, valine and hydroxyproline or a mixture thereof at a final concentration of about 0.15 M to about 0.45 M; polysorbate 80 (PS80) at a concentration between about 50 and about 200 ppm; and succinic acid at a concentration between about 1 mM and about 20 mM; wherein said lyophilized composition has a content of pentameric IgM higher than about 90% of the total IgM content, a content of IgM aggregates of less than about 1.5%, and a content of IgM oligomers of less than about 7%.

21. The composition according to claim 20, wherein said polyclonal IgM is human plasma-derived IgM.

22. The composition according to claim 20, wherein prior to lyophilization the pH of the initial aqueous solution comprising IgM is between about 3.8 and about 4.5.

23. The composition according to claim 20, wherein prior to lyophilization said composition has an IgM concentration between about 15 mg/mL and about 40 mg/mL.

24. The composition according to claim 20, wherein prior to lyophilization in said composition polyclonal IgM is at least about 95% by weight of the total weight of the composition.

25. The composition according to claim 20, wherein prior to lyophilization in said composition the amino acid is proline, a mixture of glycine and alanine, a mixture of proline and glycine or a mixture of proline and alanine.

26. The composition according to claim 25, wherein said mixture of glycine and alanine is an equimolar mixture of glycine and alanine.

27. The composition according to claim 25, wherein said mixture of glycine and alanine is an equimolar mixture of proline and glycine.

28. The composition according to claim 25, wherein said mixture of glycine and alanine is an equimolar mixture of proline and alanine.

29. The composition according to claim 20, wherein prior to lyophilization in said composition the final concentration of the amino acid is between about 0.17 M and about 0.43 M.

30. The composition according to claim 20, wherein prior to lyophilization in said composition the final concentration of PS80 is between about 55 ppm and about 190 ppm.

31. The composition according to claim 20, wherein prior to lyophilization in said composition the final concentration of succinic acid is between about 2 mM and about 15 mM.

32. The composition according to claim 20, wherein prior to lyophilization trehalose is further added at a concentration between about 5 mM and about 35 mM.

33. The composition according to claim 20, wherein said lyophilized composition has a content of pentameric IgM higher than about 95% of the total IgM content.

34. The composition according to claim 20, wherein said lyophilized composition has a content of IgM aggregates of less than about 1.0%.

35. The composition according to claim 20, wherein said lyophilized composition has a content of IgM oligomers of less than about 5%.

36. The composition according to claim 20, wherein said lyophilized composition is stable at least during 6 months.

37. The composition according to claim 20, wherein said lyophilized composition has a reconstitution time of less than 10 minutes.
